(19) United States
(12) Patent Application Publication
(10) Pub. No.: US 2021/0401388 A1
(43) Pub. Date: Dec. 30, 2021

(54) System for Hybrid Positron Emission Tomography/Computed Tomography

(71) Applicant: Siemens Medical Solutions USA, Inc., Malvern, PA (US)

(72) Inventors: Inki Hong, Knoxville, TN (US); Ziad Burbar, Knoxville, TN (US); James L. Corbeil, Knoxville, TN (US)

(21) Appl. No.: 17/303,800

(22) Filed: Jun. 8, 2021

Related U.S. Application Data
(60) Provisional application No. 62/705,466, filed on Jun. 29, 2020.

(51) Int. Cl. A61B 6/00; A61B 6/03; A61B 6/04; A61B 6/10

(52) U.S. Cl. CPC A61B 6/4417; A61B 6/037; A61B 6/102; A61B 6/032; A61B 6/04

(57) ABSTRACT

A hybrid imaging system, includes a positron emission tomography (PET) scanning system including a gantry having two ends with a patient tunnel extending therethrough, a first secondary scanning system including a gantry having two ends with a patient tunnel extending therethrough and coaxially arranged with the PET scanning system. The second end of the first secondary scanning system's gantry opposes the first end of the PET scanning system's gantry and the two patient tunnels define an extended patient tunnel defined through the PET scanning system and the first secondary scanning system. A first patient handling system (PHS) is positioned adjacent to the first end of the first secondary scanning system's gantry to carry a first patient into the extended patient tunnel, and a second PHS is positioned adjacent to the second end of the PET scanning system's gantry to carry a second patient into the extended patient tunnel.

### CROSS REFERENCE TO RELATED APPLICATIONS

This application claims the benefit of U.S. Provisional Patent Application No. 62/705,466, filed Jun. 29, 2020, which is hereby incorporated by reference for all purposes as if fully set forth herein.

### BACKGROUND

### 1. Field of the Disclosure

The present disclosure relates to medical imaging. More specifically, the present disclosure relates to system configuration for throughput optimization of a long axial field of view combined positron emission tomography/computerized tomography hybrid medical imaging systems.

### 2. Description of the Related Art

Systems for medical imaging use techniques to get detailed images of the body for diagnostic purposes. Positron emission tomography (PET) is an imaging technique that uses radioactive substances known as radiotracers to visualize and measure changes in metabolic processes, and in other physiological activities including blood flow, regional chemical composition, and absorption. Computed tomography (CT) is a noninvasive medical imaging technique. A CT scan combines a series of X-ray images taken from different angles around a patient and uses computer processing to create cross-sectional images (slices) of the bones, blood vessels and soft tissues.

In recent years, PET systems have improved their sensitivity performances by improving the Time of Flight (ToF) and improving the solid angle by elongating the axial field of view (FoV). Time of flight (ToF) is the measurement of the time taken by a particle or electromagnetic wave to travel a distance through a medium. Time-of-flight PET makes use of very fast gamma-ray detectors and data processing system that can more precisely determine the difference in time between the detection of two photons. The benefits of long axial FOV systems allow a whole-body scan and higher sensitivity. Generally, PET scanners with axial FOV longer than about 22 cm are referred to as long axial FOV PET scanners.

PET scans are increasingly read alongside CT scans providing both anatomic and metabolic information (i.e., what the structure is, and what it is doing biochemically). PET scanners are now available with integrated high-end multi-detector-row CT scanners (so-called “PET-CT”). Because the two scans can be performed in immediate sequence during the same session, with the patient not changing position between the two types of scans, the two sets of images are more precisely registered, so that areas of abnormality on the PET imaging can be more perfectly correlated with anatomy on the CT images. This is very useful in showing detailed views of moving organs or structures with higher anatomical variation, which is more common outside the brain.

However, the price of a long axial FoV PET system is very high. Thus, it is cost beneficial for the system to scan as many patients per day as possible. With improved sensitivity performance over prior systems such as a short axial FoV PET system, this allows the patient scan time to be reduced. Currently system users have taken advantage of the higher sensitivity by either reducing the scan time with the same radiotracer injection, or reducing the dosage in the injection and maintaining a similar scan time. Even with some time savings, the preparation and loading and unloading of the patient on the scanner bed becomes the limiting factor to have a higher patient throughput.

### SUMMARY OF THE DISCLOSURE

To overcome the problems described above, preferred embodiments of the present disclosure provide an imaging system with a CT and a patient handling system (PHS) on both sides of a PET system. As such, the PET system can be in the center and two scan rooms can be provided adjacent to each other with the PET system in between.

A hybrid imaging system, includes a positron emission tomography (PET) scanning system including a gantry having a first end and a second end with a patient tunnel extending therethrough extending from the first end to the second end; a first secondary scanning system including a gantry having a first end and a second end with a patient tunnel extending therethrough and coaxially arranged with the PET scanning system at the first end of the PET scanning system, wherein the second end of the first secondary scanning system's gantry opposes the first end of the PET scanning system's gantry and the two patient tunnels define an extended patient tunnel defined through the PET scanning system and the first secondary scanning system; a first patient handling system (PHS) positioned adjacent to the first end of the first secondary scanning system's gantry and configured to carry a first patient into the extended patient tunnel; and a second PHS positioned adjacent to the second end of the PET scanning system's gantry and configured to carry a second patient into the extended patient tunnel.

The hybrid imaging system can further include a second secondary scanning system including a gantry having a first end and a second end with a patient tunnel extending therethrough and coaxially arranged with the PET scanning system at the second end of the PET scanning system and positioned between the PET scanning system and the second PHS, whereby the second secondary scanning system's gantry's patient tunnel further extends the extended patient tunnel and the second PHS is configured to carry the second patient into the extended patient tunnel.

The hybrid imaging system where first secondary scanning system is a computed tomography (CT) scanning system. The hybrid imaging system where at least one of the first secondary scanning system and the second secondary scanning system is a computed tomography (CT) scanning systems.

The hybrid imaging system where the first secondary scanning system is a single photon emission computed tomography (SPECT) scanning system. The hybrid imaging system where at least one of the first secondary scanning system and the second secondary scanning system is a single photon emission computed tomography (SPECT) scanning system.

The hybrid imaging system where the PET scanning system is a long axial field of view scanner or a is a short axial field of view scanner.

A method of imaging using a hybrid imaging system includes scanning the first patient with the first secondary scanning system and the PET scanning system; and scanning the second patient with the PET scanning system.

The method of claim 9 can further include when either the scanning of the first patient or the scanning of the second patient involves fully extending the respective PHS into the PET scanning system, moving the other PHS away from the extended patient tunnel to avoid collision between the two PHS.

The method further includes when either the scanning of the first patient or the scanning of the second patient involves fully extending the respective PHS into the PET scanning system, moving the other PHS away from the extended patient tunnel to avoid collision between the two PHS. The method of where the first secondary scanning system is a first computerized tomography (CT) scanning system; and the second secondary scanning system is a second CT scanning system.

A command and motion control system for an imaging system including a positron emission tomography (PET) scanning system, a first secondary scanning system, and a second secondary scanning system, the command and motion control system includes an acquisition system that collects data and processes an image from the PET scanning system, the first secondary scanning system, and the second secondary scanning system; and a motion control system that provides motion commands to a first patient handling system (PHS) for the first secondary scanning system and a second PHS for the second secondary scanning system.

The command and motion control system can further include an operator console to operate the PET scanning system and the first secondary scanning system, and/or the second secondary scanning system.

The command and motion control system can further include a switch that controls communication between nodes in the command and motion control system. The communication can include any combination of data, commands, and power.

The command and motion control system where the switch communicates directly with any of the acquisition system, the first secondary scanning system, the second secondary scanning system, the motion control system, the first PHS, and the second PHS.

The command and motion control system where the motion control system provides motion control and safety protocol of patient motions in the imaging system.

The above and other features, elements, characteristics, steps, and advantages of the present invention will become more apparent from the following detailed description of preferred embodiments of the present invention with reference to the attached drawings.

### BRIEF DESCRIPTION OF THE DRAWINGS

FIG. 1 is a block diagram of an imaging system according to the current disclosure.

FIG. 2 is a block diagram of a command and motion control system architecture according to the current disclosure.

### DETAILED DESCRIPTION OF PREFERRED EMBODIMENTS

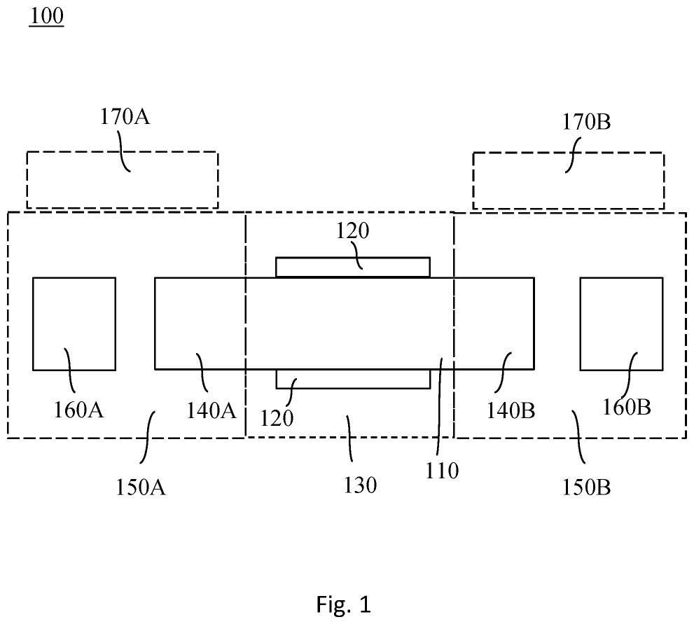

FIG. 1 is a block diagram of an imaging system 100 according to the current disclosure. FIG. 1 shows a long axis FoV PET system 110 with a PET detector 120 located in a central space 130. FIG. 1 also show two secondary scanning systems, CT systems 140A and 140B, arranged to be on opposite sides of the PET system 110 and respectively located in separate scan spaces 150A and 150B. The combination of the PET system 110 and the first CT system 140A form one PET/CT hybrid scanner system and the combination of the PET system 110 and the second CT system 140B form another PET/CT hybrid scanner system. Optionally, a Single Photon Emission Computed Tomography (SPECT) system can be used as one or both of the secondary scanning systems.

Further, a PHS 160A and 160B associated with each of the two PET/CT hybrid systems can be located in each of the respective spaces 150A and 150B. The purpose of the PHS is to facilitate acquisition of patient images, manage patient motion, ensure geometric accuracy, and control workflow through the imaging system. A PHS can include a treatment table that provides patient support and comfort during the imaging process and a shuttle to move the patient within the facility. Additionally, an operator space 170A and 170B associated with each PET/CT hybrid systems and corresponding PHS 160A, 160B can be located adjacent to each space 150A, 150B, respectively. In this arrangement, the PET system 110 can be centrally located with the PET system 110 and two scan spaces 150A, 150B oriented in line with each other. This results in effectively two working PET/CT hybrid systems that are coaxially arranged to share one PET system 110 hardware. Because one PET system 110 is shared, the two secondary scanning systems 140A, 140B and the PET system 110 need to be coaxially arranged to accommodate proper movements of the patients on the PHS 160A, 160B that are axially aligned with the shared PET system 110 and the two secondary scanning systems 140A, 140B. This results in a cost effective two PET/CT hybrid system with the throughput equivalent of two separate PET/CT hybrid systems. Two patients, each on their respective PHS 160A and 160B, can be scanned serially by the two PET/CT hybrid system. Time efficiency can be achieved because a second patient can be prepared for a scan in the second scan space 150B while the first patient is being scanned in the first scan space 150A.

This arrangement can be accomplished with any of a number of configurations. For example, the spaces 130, 150A, and 150B can be one long room that includes two CT systems 140A, 140B, two PHSs 160A, 160B, and one PET system 110. This one room can be separated in the middle by a radiation shield of glass or another material, or any type of room separator so that when the PET/CT hybrid system on one side of the room is in a scanning session with a first patient, a technician and a second patient can be safely in the other side of the room preparing the second patient for a next scan session with the second PET/CT hybrid system. Alternatively, the space can be built as two rooms adjacent to each other where the center of the PET system 110 is in the opening of a wall between the two rooms. Alternatively, the configuration can include three rooms, two rooms with the CT systems 140A, 140B and PHSs 160A, 160B, and a utility room in between. In such a configuration, the PET system 110 and a portion of both of the CT systems 140A, 140B can be in the utility room with the remainder of the CT systems 140A, 140B extending through separate walls into the outer rooms.

Depending on the FoV length of the PET system 110 and the size of a PHS 160A, 160B, a moving mechanism (not shown) can be included with each of the PHS 160A, 160B to move the respective PHS away from the corresponding CT system 140A, 140B so that each of the two opposing PHS have the option to fully extend into the gantry of the shared PET system 110. The moving mechanism to move each PHS is in place to relocate the PHS away from their corresponding PET/CT hybrid system to avoid any collision with the opposing PHS that is extending to its fully extended position in the gantry of the PET system 110. For example, when the first PHS 160A moves through the first CT system 140A and fully extends into the gantry and patient tunnel of the shared PET system 110, the leading end of the fully extending first PHS 160A will move through and beyond the gantry of the PET system 110 and extend into the second CT system 140B (the second secondary scanning system). If the second PHS 160B is not cleared out of the second CT system 140B, the two PHS 160A and 160B could collide.

As a result, a hybrid imaging system can include the PET system 110 including a gantry with two ends and a patient tunnel extending from end to end and the first CT system 140A including a gantry with two ends and a patient tunnel extending end to end that is coaxially arranged at one end of the PET system 110. Thus, one end of the gantry of the first CT system 140A opposes one end of the gantry of the PET system 110 and the two adjacent patient tunnels of the PET system 110 and the first CT system 140A define an extended patient tunnel defined through the combined PET/CT hybrid scanning system. Additionally, the PHS 160A can be positioned adjacent to one end of the gantry of the first CT scanning system 140A to carry a first patient into the extended patient tunnel. The second PHS 160B can be adjacent to the other end of gantry of the PET system 110 and carry a second patient into the extended patient tunnel from the other direction.

Optionally, the hybrid imaging system can further include the second CT system 140B that includes a gantry with two ends and a patient tunnel extending therethrough and also coaxially arranged with the PET system 110 at another end of the PET system 110 and between the PET system 110 and the second PHS 160B. In this arrangement, the gantry of the second CT system 140B and corresponding patient tunnel further extends the extended patient tunnel and the second PHS 160B carries the second patient into the extended patient tunnel. In operation, when either scanning of the first patient or the scanning of the second patient involves fully extending the respective PHS into the PET system 110, the other PHS is moved away from the extended patient tunnel to avoid collision between the two PHSs.

As shown in FIG. 1, the imaging system 100 can include two separate operator spaces 170A, 170B. This allows for continuous monitoring by separate PET system operators of two patients during patient preparation, scanning, and unloading after the scan. Alternatively, there can be one operator space that is configured so that one operator can monitor the patients. Alternatively, a camera system can be used to feed a continuous live feed to an operator to maintain continuous monitoring of the patients.

For illustration, a scan with a long axis FoV PET system can take 2-3 minutes with a patient preparation time of 7-10 minutes. Thus, while a first patient is being scanned in CT system 140A in space 150A, a second patient can be prepared for a scan with CT system 140B in space 150B. Patient preparation can include orientation, isotope infusion, and connection of monitoring devices, i.e., ECG and/or respiratory devices connections. Once the scan of the first patient in space 150A is completed, a scan of the second patient in space 150B can begin. In the meantime, the first patient in space 150A can be unloaded from the PHS 160A and a third patient can be brought in for scan preparation in space 150B.

As the imaging system 100 includes two CT systems 140A, 140B this introduces a great deal of complexity to the motion control system. The control system has to be configured to adapt to two PHS, two CT systems, and one PET system. As a result, there likely will be two computer stations, one for each side of the imaging system 100.

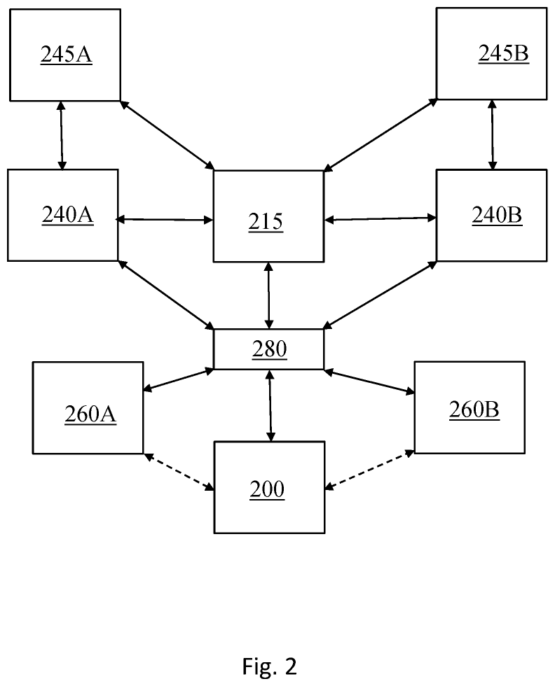

FIG. 2 is a block diagram of a command and motion control system architecture according to the current disclosure. Each block represents hardware or circuitry and/or software that perform certain functions and the arrows represent communication paths between the hardware. The dashed line arrows indicate motion commands and the solid line arrows indicate operation commands and data flow.

FIG. 2 shows that the command and motion control system can include a motion control system 200 that communicated with each PHS 260A and 260B to arbitrate the motion commands and priority for each automated PHS motion. The command and motion control system can include lock/unlock logic to determine which system is moving a PHS 260A, 260B as both a CT-A 240A and CT-B 240B and the PET can move a PHS. The diagram of FIG. 2 is a mix of deployment and logical views. The operator consoles 245A and 245B of each respective CT 240A, 240B can, for example, be located on the same computer system in one operator room, and cameras can be provided to monitor the patients. Optionally, the operator consoles 254A, 245B can be located on separate computer systems in separate operator rooms, and the control logic can provide status including when it is ready to scan. A patient list can be queued to scan the next patient on systems A or B.

FIG. 2 shows that the command and motion control system can include a switch 280. The switch 280 can be a communication hub that regulates and passes communication to nodes in the system. The communication can include any combination of data, commands, and power. The switch 280 can be a network switch and can be implemented via hardware and/or software. As shown, the switch 280 can direct communications to the motion control system 200, the PHSs 260A, 260B, the CTs 240a, 240B, and a PET system acquisition station 215. The switch can be located as a portion of the motion control system that hosts the motion control and safety protocols of the motions.

The motion control system can be set to accommodate the image plane separation between each CT and the PET system to located and relocate the PHSs 26A, 260B. Furthermore, the PET acquisition system 215, which provides data capture and image processing and reconstruction, must accommodate its image plane orientation to each CT. In a conventional PET/CT hybrid system, the first detector ring is toward the CT and the last detector ring is toward the back of the PET system. Because the present embodiment includes two CT systems 240A and 240B, one of the CT systems is located at the back of the PET system. Thus, an image from one PET/CT system of the present embodiment will have the same image constructions as a conventional PET/CT. However, an image from the other PET/CT of the present embodiment will be flipped. Thus, the image of one PET/CT of the present embodiment needs to be reoriented to the correction orientation during image processing and formation. That is, the transformation matrix, a mathematical representation of the 3-D image of the patient as the patient is moved through the imaging system, needs to flip the PET image 180 degrees to match the CT image.

Although the current embodiment is directed to a CT/PET/CT system, it should be understood that similar arrangements could be beneficial for other types or styles of imaging systems that could include short axis field of view PET systems, Single Photon Emission Computed Tomography (SPECT), or any other suitable combination of imaging systems.

It should be understood that the foregoing description is only illustrative of the present invention. Various alternatives and modifications can be devised by those skilled in the art without departing from the present invention. Accordingly, the present invention is intended to embrace all such alternatives, modifications, and variances that fall within the scope of the appended claims.

What is claimed is:

1. A hybrid imaging system, comprising:
a positron emission tomography (PET) scanning system including a gantry having a first end and a second end with a patient tunnel extending therethrough extending from the first end to the second end;
a first secondary scanning system including a gantry having a first end and a second end with a patient tunnel extending therethrough and coaxially arranged with the PET scanning system at the first end of the PET scanning system, wherein the second end of the first secondary scanning system's gantry opposes the first end of the PET scanning system's gantry and the two patient tunnels define an extended patient tunnel defined through the PET scanning system and the first secondary scanning system;
a first patient handling system (PHS) positioned adjacent to the first end of the first secondary scanning system's gantry and configured to carry a first patient into the extended patient tunnel; and
a second PHS positioned adjacent to the second end of the PET scanning system's gantry and configured to carry a second patient into the extended patient tunnel.

2. The hybrid imaging system of claim 1, further comprising a second secondary scanning system including a gantry having a first end and a second end with a patient tunnel extending therethrough and coaxially arranged with the PET scanning system at the second end of the PET scanning system and positioned between the PET scanning system and the second PHS, whereby the second secondary scanning system's gantry's patient tunnel further extends the extended patient tunnel and the second PHS is configured to carry the second patient into the extended patient tunnel.

3. The hybrid imaging system of claim 1, wherein the first secondary scanning system is a computed tomography (CT) scanning system.

4. The hybrid imaging system of claim 2, wherein at least one of the first secondary scanning system and the second secondary scanning system is a computed tomography (CT) scanning systems.

5. The hybrid imaging system of claim 1, wherein the first secondary scanning system is a single photon emission computed tomography (SPECT) scanning system.

6. The hybrid imaging system of claim 2, wherein at least one of the first secondary scanning system and the second secondary scanning system is a single photon emission computed tomography (SPECT) scanning system.

7. The hybrid imaging system of claim 1, wherein the PET scanning system is a long axial field of view scanner.

8. The hybrid imaging system of claim 1, wherein the PET scanning system is a short axial field of view scanner.

9. A method of imaging using a hybrid imaging system of claim 1, the method comprising:
scanning the first patient with the first secondary scanning system and the PET scanning system; and
scanning the second patient with the PET scanning system.

10. The method of claim 9, further comprising when either the scanning of the first patient or the scanning of the second patient involves fully extending the respective PHS into the PET scanning system, moving the other PHS away from the extended patient tunnel to avoid collision between the two PHS.

11. The method of claim 9, wherein the first secondary scanning system is a first computerized tomography (CT) scanning system.

12. A method of imaging using a hybrid imaging system of claim 2, the method comprising:
scanning the first patient with the first secondary scanning system and the PET scanning system; and
scanning the second patient with the second secondary scanning system and the PET scanning system.

13. The method of claim 12, further comprising when either the scanning of the first patient or the scanning of the second patient involves fully extending the respective PHS into the PET scanning system, moving the other PHS away from the extended patient tunnel to avoid collision between the two PHS.

14. The method of claim 12, wherein
the first secondary scanning system is a first computerized tomography (CT) scanning system; and
the second secondary scanning system is a second CT scanning system.

15. A command and motion control system for an imaging system including a positron emission tomography (PET) scanning system, a first secondary scanning system, and a second secondary scanning system, the command and motion control system comprising:
an acquisition system that collects data and processes an image from the PET scanning system, the first secondary scanning system, and the second secondary scanning system; and
a motion control system that provides motion commands to a first patient handling system (PHS) for the first secondary scanning system and a second PHS for the second secondary scanning system.

16. The command and motion control system of claim 15, further comprising an operator console to operate the PET scanning system and the first secondary scanning system, and/or the second secondary scanning system.

17. The command and motion control system of claim 15, further comprising a switch that controls communication between nodes in the command and motion control system.

18. The command and motion control system of claim 17, wherein the communication includes any combination of data, commands, and power.

19. The command and motion control system of claim 17, wherein the switch communicates directly with any of the acquisition system, the first secondary scanning system, the second secondary scanning system, the motion control system, the first PHS, and the second PHS.

20. The command and motion control system of claim 15, wherein the motion control system provides motion control and safety protocol of patient motions in the imaging system.

21. The command and motion control system of claim 15, wherein the PET scanning system is a long axial field of view scanner.

22. The command and motion control system of claim 15, wherein the first secondary scanning system and the second secondary scanning system are computed tomography (CT) scanning systems.

23. The command and motion control system of claim 15, wherein the first secondary scanning system and the second secondary scanning system are single photon emission computed tomography (SPECT) scanning systems.
